(19) United States
(12) Patent Application Publication
(10) Pub. No.: US 2005/0226905 A1
(43) Pub. Date: Oct. 13, 2005

(54) Biocompatible compositions as carriers or excipients for pharmaceutical and nutraceutical formulations and for food protection

(76) Inventors: Canh Le Tien, Quebec (CA); Monique Lacroix, Quebec (CA); Mircea Alexandru Mateescu, Quebec (CA); Pompilia Ispas-Szabo, Quebec (CA)

Correspondence Address: Norman H Zivin;Cooper & Dunham, 1185 Avenue of the Americas, New York, NY, 10036, US

(21) Appl. No.: 10/478,468

(22) PCT Filed: May 23, 2001
(86) PCT No.: PCT/CA01/00726

§ 371 Date: Apr. 25, 2005

(30) Foreign Application Priority Data
May 22, 2001 (CA) 2,248,799

(51) Int. Cl. A61K 31/734; A61K 31/722; A61K 47/00

(52) U.S. Cl. 424/439; 514/54; 514/55

(57) ABSTRACT

This invention refers to biocompatible carbohydrate polymers such as modified polysaccharides (e.g. chitosan, alginate), associated with milk protein (e.g. caseinate and/or whey proteins) designed to carry bioactive agents. The formulations may be used in various delivery systems including beads, tablets, microencapsulating agents and coatings for oral dosage forms, implants for subcutaneous devices and films for topic administration and food protection. These formulations present improved chemical resistance and exert their activity for prolonged time into gastrointestinal tract (GIT) and blood circulation as well as for preserving food qualities over long period. The association of modified chitosan, modified alginate with milk proteins results in a stabilized structure able to control the release of drugs, bacteria, bacteriocines, enzymes, nutraceutics, etc. into enteric, topic or systemic route.

### CROSS REFERENCE TO RELATED APPLICATIONS

This is the National Phase of International Application No. PCT/CA01/00726, filed May 23, 2001, which was published in English under PCT Article 21(2), and which is incorporated herein in its entirety.

### FIELD OF INVENTION

The present invention is related to carriers or excipients for bioactive agents, for example, as carriers or excipients for formulation of pharmaceutics or nutraceutics for biomedical or biotherapeutic applications or for food protection.

### BACKGROUND OF THE INVENTION

The usefulness of specific polymers in drug delivery systems is well established. Numerous polymers, available as such or adequately modified, are intensively used as main components of drug controlled release systems, which can be classified into four major categories: (1) diffusion controlled systems, (2) solvent activated (swelling) systems, (3) chemically controlled systems, and (4) magnetically controlled systems.

More specifically, the term “controlled release delivery systems” means a drug or bioactive agent delivery controlled by the polymeric matrix and, by time or location or by both time and location. That system is designed to allow the release of the contents at a controlled time, following a controlled time, and at a desired site (systemic circulation or particular location).

Such systems have been developed in the past for immediate release of a certain, well-determined dose and later for maintenance of the concentration over an extended period of time (N. A. Peppas, Hydrogels in Medicine and Pharmacy, Academic Press, 1987; V. Ranade and M. A. Hollinger, Drug Delivery systems, CRS Press, Boca Raton, 1996).

Pharmaceutical formulations can be presented in a wide range of forms such as granules or beads, membranes, tablets, implants etc., each form being related to: i) the route of administration; ii) the characteristics of the bioactive agent (quantity, solubility) and of the polymer(s); and, iii) the release mechanism, the site of action, etc.

Pharmaceutical, nutraceutical or food formulations can be presented as microparticles, films, coating, microencapsulation, etc. and can be related to the type of application, the solubility, the polymers involved, the medium used, the polymer functionality and the release mechanism.

There are several types of dosage forms (microspheres for blood circulation, liposomes, capsules and tablets for oral administration, implants, transdermal patches, suppositories for rectal and vaginal delivery, ophthalmic fluids, etc).

Oral administration is the way preferred for delivery of active agents absorbable by intestinal wall.

Although capsules and osmotic devices (Alza's OROS™ system, cited by Ranade and Hollinger, 1996; V. Ranade, J. Clin. Pharmacol. 31, 2, 1991) allow good drug release kinetic profiles, manufacturers prefer, when possible, monolithic devices (e.g. tablets and implants) since they are easier to formulate.

Implants represent a pharmaceutical formulation for drugs (which cannot be administered orally because they are not adsorbed by intestinal wall), that can be delivered directly in the blood stream. There is a growing interest for such formulation, particularly for delivery of steroids, antibiotics, analgesics, chemotherapeutics, insulin, etc). Implants are placed completely under the skin, for long chronic periods or for only transient therapy (thus, the implant can be removed after a desired time).

Matrix systems (monolithic devices) have some major advantages relative to other types of controlled release drug and bioactive agent delivery systems, for example, the ease of manufacture. In general, matrix devices can be prepared by mixing the drug as a finely divided powder with the polymeric excipient. This mixture is then placed in an appropriate mould (die) of a compression device and the resultant tablets are ready to use. Among the variety of controlled release devices, the following are frequently used: (1) dissolved systems that are prepared from a matrix containing a drug at or below the saturation solubility of the drug in the polymer; (2) dispersed systems that contain the drug within a matrix at a concentration that greatly exceeds the saturation stability of the drug in the polymer; (3) reservoir-dispersed matrix systems that are analogous to the dispersed system except that a barrier layer is present at the surface of the device which is of lower permeability to the drug than the bulk polymer; and (4) porous matrix systems that are prepared from a dispersion of drug particles and pre-formed polymer. In porous matrix systems, it is assumed that upon leaching of the drug, continuous macroscopic pores or channels arise from the displacement of drug by solvent.

In monolithic systems the drug is physically incorporated into a polymer matrix and is released to the surrounding environment as the polymer bioerodes. If mobility of the drug in the matrix is such that rapid diffusion release is possible, its dissolution kinetics will be first order. Zero-order release requires an erosion process confined to the surface of the solid device and the drug highly immobilized into the matrix. Although surface erosion is difficult to achieve, such systems have several significant advantages, for example, the ability to control drug delivery rate by simply varying drug or bioactive agents loading within the matrix, controlling lifetime of the device, varying the physical dimension of the device, and the ability of one matrix to deliver a variety of therapeutic agents.

Alternatively, or in combination, a coating may be applied. Such coating can be dissolved under specific ionic (i.e. acidic) conditions, delivering the contained bioactive agent at a desired destination. For example, the coating may be dissolved in acidic conditions for delivery in the stomach. Such coatings, which are insoluble in neutral pH (i.e. in the mouth) and soluble in acidic pH, are able to provide specific delivery to the stomach (i.e. Eudragit™ E series of copolymers poly(butyl-methacrylate), (2-dimethyl-aminoethyl)methacrylate, methyl methacrylate, ethyl caprylate (Sheu and Rosenberg J. Food Science. (1995) 60(1): 98-103). The enteric coatings are able to dissolve specifically in the small or in large intestine. Examples of such enteric coatings used in the prior art (Tsai et al., 1998 J. Controlled Release 51, 289-299.) are cellulose acetate phthalate, hydroxymethylpropyl cellulose (HPMC), and polymethacrylates (Eudragit™ L and S series of copolymers).

Numerous polymeric excipients have been identified as compatible with controlled delivery of drugs and bioactive agents administered enterically or systemically. These polymeric forms include microparticles, hydrogel, self-diffusion and self-regulated systems, biodegradable polymers and porous membranes. Hydrogel systems were first used for the delivery of insulin in diabetic rat models (Davis, S K, Experientia, (Mar. 15, 1972) 28(3):348-353), providing an aqueous microenvironment for the diffusion migration of macromolecular active agent. These gels limit the migration of bioactive agent with a release dependent on the polymer content of the gel and on the molecular weight of the encapsulated substrate (Jhon, Miss. and J D Andrade, J. Biomed. Mater. Res., (November, 1973) 7(6):509-22).

A common aspect to all beads or particles is the difficulty to keep the biologically active compounds inside the matrix as the biologically active compounds are usually made of materials that permeate the microparticles, therefore being released before reaching the selected target site. So far, the problem of keeping the biologically active compounds within the microparticles has been mainly solved by the modification of the structure, especially the walls of the particles, rendering them less permeable to their bioactive agent load. However, such an approach may induce the loss, in part, of the physical-chemical characteristics of the particles, due to the changes in the structure. Considering that such carriers should be developed specifically for each biologically active compound to be used, the process of manufacturing microparticles containing biologically active compounds can become very expensive.

Alternatively, the present invention proposes formulations with a large versatility, allowing a good biocompatibility with various types of bioactive agents to be transported.

Biodegradable microspheres have been successfully used to deliver drugs at a controlled rate to specific tissues (e.g. the brain (Cohen, S., et al., Pharm. Res. (June, 1991) 8(6):713-20, and, Walter, Kans., et al, Cancer Res. (Apr. 15, 1994) 54(8):2207-12).

The long-term goal for the encapsulation of bioactive agents for intestinal microbial equilibration, nutraceutical application, immunostimulation, antitumor, anti-inflammatory or other therapies is to provide sustained local release. Such formulations ideally contain concentrated bioactive agents in acceptable volumes for delivery, inducing minimal tissue reaction to the polymer.

There is a growing interest in the chemical modification of polysaccharides, such as chitosan and alginate, as they have a large potential of providing new applications for such abundant polymers.

Milk proteins act as film forming agents and, together with cellulose, form a matrix support resistant to various pH levels and proteolytic media (commonly assigned PCT application number PCT/CA00/01386 filed on Nov. 24, 2000, and, Le Tien et al, J. Agric. Food Chem., 2000, 48:5566-5575).

Several prior art patents relate to the use of chitosan in forming complexes with drugs for delivery systems.

U.S. Pat. No. 5,900,408 issued on May 4, 1999 to Block and Sables, discloses methods of creating a unique chitosan and employing the same to form dosage forms.

Nordquist et al (U.S. Pat. No. 5,747,475 issued on May 5, 1998) describes chitosan modified by the addition of a monosaccharide or oligosaccharide side chain to its free amino groups. The “glycated chitosan” preferred embodiment is a galactose derivative of chitosan useful as an immunoadjuvant in laser/sensitizer assisted immunotherapy.

El Ghaouth et al (U.S. Pat. No. 5,633,025 issued on May 27, 1997) proposes a bioactive coating for harvested commodities (a coating for harvested agricultural commodities which delays ripening and controls decay). The coating comprises a modified chitosan matrix containing a yeast antagonistic to postharvest pathogens. The modified chitosan may be carboxymethylchitosan or glycolchitosan.

Nakamura (JP 8196461 A2 published in 1996) proposes an antibacterial wipe with antiseptic properties effective on the wiped part, using modified chitosan and collagen modified with fatty acids.

Aiba (JP 62288602 A2 published in 1987) describes the production of modified chitosan particles useful as a metal capturing agent, drug sustained release carrier, enzyme immobilizing carrier, etc., obtained by dripping an acidic aqueous solution of chitosan into an alkaline aqueous medium and reacting the washed particle with a modifying reagent, e.g. acetic acid, phosphorus pentoxide, acetaldehyde, etc.

Shiotani et al (JP 3289961 A2 published in 1991) describes a wound covering material with the ability to stop bleeding and to control moisture content and vaporization. A chitosan derivative produced by chemically modifying chitosan, especially N-succinylated chitosan can be used as a medical material. Further, by combining a chitosan derivative, practical use is clinically carried out. This constitution provides adhesion, flexibility, durability and simplicity of handling.

K. Tomihata and Y. Ikada, “In vitro and in vivo degradation of films of chitin and its deacetylated derivatives”, Biomaterials, 18, 567-575 (1997) discloses chitin deacetylated with NaOH to obtain partially deacetylated chitins. The specimens used were deacetylated by 0 (chitin), 68.8, 73.3, 84.0, 90.1 and 100 mol % (chitosan). Films were prepared by casting solutions of these specimens. In vivo degradation was studied by subcutaneously implanting the films in the back of rats. Interestingly, the tissue reaction towards highly deacetylated derivatives including chitosan was very mild.

Films prepared from chitosan and alginate are potential candidates for buccal drug delivery—oral mucoadhesive films.

Chitosan was reported to form compositions with a variety of anionic drugs and polyanions such as indomethacin, polyacrylate, pectin, alginate, and some polysaccharides (J. Kristl et al, Hydrocolloids and Gels of Chitosan as Drugs Carriers, Int. J. Pharm., 99, 13-19, (1993); T. Nagai et al., Application of Chitin and Chitosan to Pharmaceutical Preparations. In “Chitin, Chitosan and Related Enzymes” Academic Press, New York, 1984, 21-39; T. Takahasbi et al., Characterization of Polyion Complex of Chitosan with Sodium Alginate and Sodium Polyacrylate. Int. J. Pharm., 61, 35-41, 1990; C. Thomas et al., Evaluation of modified alginate-chitosan polyethylene glycol microparticles for cell encapsulation, Artif. Organs, 23, 894-903, 1999; M. L. Rowsen et al., b-Cyclodextrin-insulin-encapsulated chitosan/alginate matrix: oral delivery system, J. Appl. Polym. Sci, 75, 1089-1096, 2000).

Chitosan has also been proposed for use as a biomedical membrane or artificial skin for delivery of anti-cancer drugs to tumour cells, and as a pharmaceutical delivery system. In addition, chitosan has been shown to be biodegradable, to be biocompatible, to have very low toxicity, and to have no thrombogenic activity (R. Muzzarelli, “In vivo biochemical significance of chitin-based medical items in Polymeric Biomaterials, S. Dumitriu, ed., 1994; Marcel Decker. Inc., New York).

Polysulfated chitosan derivatives, with a substitution degree by sulfur from 0.62 to 1.86, injected intravenously are known to show heparin-like action. The anticoagulant activity of chitosan derivatives depended on the degree of polymerization and sulfonation.

Chitosan has been selectively N-acylated with various carboxylic anhydrides, i.e. acetic, propionic, n-butyric, n-valeric and n-hexanoic anhydrides (K. Y. Lee et al., Blood compatibility of partially N-acylated chitosan derivatives, Biomaterials, 16, 1211-1216, 1995). N-acyl chitosans showed more blood compatible properties than N-acetyl chitosan and, in particular, N-hexanoyl chitosan was the most blood compatible. Chitosans substituted with alkyl chains having minimum six carbon atoms demonstrated hydrophobic interaction in solution. The chemical structure of synthesized polymers was studied in relation to the nature of hydrophobic chain and substitution degree, (J. Desbrieres et al., Hydrophobic derivatives of chitosans: Characterization and Theological behavior, Int. J. Biol. Macromol., 19, 21-28, (1996)). The field of application (heparin-like) is different from the drug delivery systems of the present invention.

### SUMMARY OF THE INVENTION

There is provided a biocompatible carrier composition comprising a biocompatible carbohydrate polymer in association with a milk protein.

There is also provided a biocompatible carrier composition comprising a fatty acid modified chitosan or alginate.

The compositions of the present invention are useful as carriers or excipients for bioactive agents. The compositions are particularly useful in controlled release delivery systems for bioactive agents and for immobilizing bioactive agents.

### DETAILED DESCRIPTION OF THE INVENTION

In a preferred aspect, the biocompatible carbohydrate polymer is hydrophobic in nature in order to reduce its solubility in aqueous systems. The hydrophobicity of the carbohydrate polymer may be enhanced by modifying it with a hydrophobic group. Polysaccharides, particularly hydrophobically modified polysaccharides, are especially preferred forms of the biocompatible carbohydrate polymer. Chitosan and alginate are more particularly preferred polysaccharides, particularly when modified with a hydrophobic group.

Hydrophobic groups used to modify the biocompatible carbohydrate polymer are groups that will reduce the solubility of the carbohydrate polymer in an aqueous environment. Such hydrophobic groups include unsubstituted or substituted alkyl or aryl groups of sufficient size to impart increased hydrophobicity to the carbohydrate polymer. Particularly useful hydrophobic groups are residues of aldehydes or fatty acids, preferably (C₃-C₁₈) fatty acids. The fatty acids may be saturated or unsaturated. Examples of such fatty acids are palmitic acid, lauric acid, oleic acid, linoleic acid, linolenic acid, caproic acid, caprylic acid, stearic acid, propionic acid and butyric acid.

Modification of the carbohydrate polymer may be accomplished by functionalizing an active site on the polymer with an active form of a compound from which a hydrophobic group is to be derived. For example, amine groups on chitosan may be functionalized by reaction with fatty acid halides. In another embodiment, hydroxyl groups on alginate may be first functionalized with ethylamine to form an ehtoxyamine side group and the amine group on the ehtoxyamine further functionalized by reaction with a fatty acid halide.

Modification of the carbohydrate polymers may also be accomplished by cross-linking. For example, dialdehydes (such as glutaraldheyde), ethylchloroformate, epichlorhydrin, phosphorus oxychloride and others may be used. A dialdheyde, particularly glutaraldehyde, is a preferred cross-linking agent. Cross-linking may be done with or without modification of the carbohydrate polymer by fatty acids or other hydrophobic groups. Carbohydrate polymers that are both modified with a fatty acid and cross-linked are particularly preferred.

Milk proteins are generally classified into casein and whey proteins, which may be present in the composition either alone or in combination. An example of a whey protein is β-lactoglobulin. Casein comprises about 80% of milk protein and consists of three major components, which are α, β and κ. Casein molecules possess an open random-coil structure exhibiting little defined secondary structure. Caseinates may be formed by acidifying casein to solubilize calcium phosphate and to release casein molecules followed by neutralization of the acid casein with alakli. Sodium, calcium magnesium and potassium caseinates may be formed in this way. Caseinates possess good properties as emulsifiers and film forming agents and are preferred milk proteins in the compositions of the present invention.

Bioactive agents are agents that have an effect on a biological system. Bioactive agents include pharmaceutics (e.g. drugs), nutraceutics (e.g. vitamins such as vitamins A, C or E, and minerals such as iron and copper ions), probiotics (e.g. bacteria such as lactic acid bacteria), proteins, bacteriocines, enzymes, anti-oxidants and anti-microbials, among others.

Compositions of the present invention show improved chemical resistance and permit a bioactive agent to exert its activity for a prolonged period of time (e.g. in the gastro-intestinal tract (GIT) and circulatory system). In food related applications, such as in packaging for example, the compositions permit bioactive agents such as anti-oxidants and anti-microbials to help preserve food qualities over longer period of time.

In one aspect of the present invention, there is provided a new controlled release delivery system that includes a biocompatible carbohydrate polymer, caseinate and/or whey proteins which serve as an emulsifying film forming agent and as an excipient or a carrier for a bioactive agent. Also provided is a method for making a controlled release delivery system by encapsulating or incorporating a bioactive agent into microparticles, tablets, implants or films based on the mentioned bio-compatible materials. In particular, a mixture of milk proteins and modified polysaccharides improves the functionality (controlled release, permeability, antioxidant properties and elasticity) profile of the polymer used for microencapsulation. Also the molecular weight (and size) of the polysaccharides influences the protein-carbohydrate interactions.

Natural polymers which form the basis of carbohydrate polymers of the present invention are useful as supports for bioactive agents as they can be formulated into different forms (spheres, films, tablets, implants, etc.) depending on the intended application and route of administration (e.g. enteric, topic or systemic). Modification of natural polymers permits the design of biocompatible polymers with desired characteristics, in particular, controlled hydration or controlled acid or proteolytic degradation. The compositions of the present invention may be used in various delivery systems including beads, tablets, microencapsulating agents and coatings for oral dosage forms, implants for subcutaneous devices and films for topic administration and food protection.

One purpose of the microparticle formulation is to minimize the undesired outflow of a biologically active compound from a microparticle, to keep its biological activity and to release it from the microparticle in delayed or even in a controlled manner. Thus, the release of the biologically active compounds can be initiated at a certain moment, in a certain delivery site of the GI tract.

Thus, a modified polysaccharide (such as chitosan and/or alginate) cross-linked and/or derivatized with fatty acids helps formulate a bioactive agent, to protect the bioactive agent from denaturing factors of the external environment, to reduce its outflow and to control the site and the rate of its release. Milk proteins in the formulation serve as emulsifying and film forming agents and to stabilize the microparticle structure. Furthermore, milk proteins such as caseinate or whey protein have several advantages, including their utility as an excellent nutritional source for the growth of lactic bacteria in the case of probiotic formulation. Also, milk proteins (particularly caseinate) are rich in calcium, which participates in reinforcing the alginate envelope by ionotropic interactions.

In a more preferred embodiment, double stabilized microparticles (based on modified chitosan and alginate) may be prepared by using caseinate and whey proteins. A core may be formed from modified chitosan, bioactive agent, calcium caseinate, whey protein isolate (WPI) and modified sodium alginate. The formation of the intramolecular and intermolecular links between carboxylic groups of alginate and calcium ions Ca²⁺ existing in the milk proteins composition improved the stability of the preparation. The bioactive agent has thus double protection, from inside and from outside of the matrix: inside by ionic gelation with Ca²⁺ ions from milk protein, and outside by ionic gelation with Ca²⁺ ions from CaCl₂ solution.

The ratio between modified chitosan/milk protein/alginate/bioactive agent can vary depending on the desired administration route and pharmaceutical formulation, such as microparticle, tablet, implant, film or coating.

A bioactive agent may be formulated by adding an aqueous solution of the bioactive to an aqueous polymeric suspension containing derivatized chitosan, whey protein and derivatized alginate. Microparticles formed in this way can range in size from about 2 microns to 200 microns diameter. Preferably, microparticles range in diameter from about 50 to 100 microns, except for injectable forms for which the diameter is ideally less than about 10 microns. Factors affecting the particle size of the microparticle include the initial concentration of the polymers and of the proteins and the method used to form the suspension. The size of the microparticles can affect distribution, pharmacokinetics, and other factors as is well known by those skilled in the art. The smaller the microparticle diameter, the greater the surface area per unit mass, hence, the faster the release rate of the encapsulated drug.

Pharmaceutical dosages forms based on modified chitosan or alginate for oral administration may be formulated. For example, dosage forms based on monolithic devices (tablets) are of high interest because they can be obtained by direct compression of dry powders of the active therapeutic or nutraceutic agent and of the modified polymeric material (carrier or excipient). These pharmaceutical forms are of interest for therapeutic molecules administrable perorally and, in most cases, absorbable via the GI tract. Within last two decades, there is a growing interest for pharmaceutical forms allowing a control of the release of drug over 12 h or 24 h. The release control is modulated by the excipient or carrier, which regulates the water access within the tablet, the matrix swelling and/or diffusion of the drug through the polymeric structure. Such excipients or carriers can have binding properties (ensuring the mechanical stability of the tablets) and also can modulate the release of the active agent.

In the case of oral administrable formulations, the addition of one or more hydrophilic excipients, such as carrageenan, carboxymethyl cellulose, etc., to the modified chitosan or alginate is possible. In these cases, release time is expected to depend on the ratio between the hydrophilic/hydrophobic components of the composition.

The use of modified polymers as matrices for controlled release may offer several interesting advantages. Firstly, derivatization with fatty acids may limit the water access within the matrix. Secondly, fatty acids can act as plasticisers, improving the mechanical properties of the polymeric matrices.

Chitosan and alginate are preferably used as matrices to protect bioactive agents from denaturing factors of the external environment, while milk proteins are preferably used as emulsifying and film forming agents. The presence of whey proteins may also create a microenvironment where a different degree of gelation is observed inside the microparticles. Native chitosan and alginate generally have filmogenic characteristics, however they are not very resistant to water. Therefore they could benefit from modifications to acquire some desired characteristics (hydrophobicity, acid-proof and satisfactory mechanical characteristics). The modifications are essentially based on coupling with a functionalizing agent (such as an acylation agent) or a cross-linking agent (such as a bifunctional cross-linking agent).

Chitosan is a polymer of animal origin obtained after partial deacetylation of chitin. The basic unit of chitosan is essentially the -glucose-2-amine unit. Generally, functionalization of chitosan occurs at the 2-amine group (NH₂) in this unit (Oyrton and Claudio, Int. J. Biol Macromol, 26, 119-128, 1999). Cross-linking is also possible using bifunctional agents such as dialdehydes, allowing the formation of intermolecular bridges between the chitosan chains.

Chitosan may be purchased commercially under the trade-mark Kitomer™. Chitosan having a viscosity of 100-300 centipoise is suited for pharmaceutic application in the formation of tablets while chitosan having a viscosity of about 4000 centipoise is better suited for forming films and spheres.

Alginate is a polysaccharide produced by the Phaeophyceae algae. It is formed from the association of two acid-based chains: alpha-D-mannuronic acid and alpha-L-guluronic acid (Haug, Rept. No. 30, Norwegian Institute Seaweed Research, Trondheim, Norway, 1964). Alginate may be purchased from Sigma™. Medium viscosity alginate is preferably used in pharmaceutic applications.

Alginate may be modified in various ways. Acylation with a fatty acid may be done directly after deprotonation of the carboxyl groups from alginate with a strong base to produce an ester. Acylation and/or cross-linking may be done after previous derivatization with ethylamine. Cross-linking may be done without acylation with a fatty acid.

### BRIEF DESCRIPTION OF THE DRAWINGS

FIG. 1 depicts a sphere constructed in accordance with the present invention for carrying a bioactive agent.

FIG. 2A depicts an FTIR spectrum of chitosan modified by fatty acid and cross-linked by dialdehyde.

FIG. 2B depicts an FTIR spectrum of unmodified and uncross-linked chitosan.

FIG. 2C depicts the structure of modified and cross-linked chitosan.

FIG. 3 is a graph depicting release control of acetaminophen from alginate-based tablets.

FIG. 4 is a graph depicting release control of acetaminophen from chitosan-based tablets.

### EXAMPLES

The mechanical properties of chitosan- and alginate-based films were analysed.

Native forms of chitosan and alginate have relatively important antioxidant properties. Their capacity to trap free radicals is between 55% and 65%. After modification, their antioxidant power is slightly diminished (5-10%). However, by the incorporation of calcium caseinate or whey protein isolate in the formula, an increase was noticed and the values were raised to 70 to 80%.

Materials based on native chitosan and alginate are highly sensitive to water and the recovery yield (RY) is very low, practically 0% (Gontard et al., J. Food Sci., 57, 190-199, 1992). Following the coupling with fatty acids and/or the cross-linking by dialdehydes, the polymers became more water-resistant and the RY values increase up to 71 and 80%.

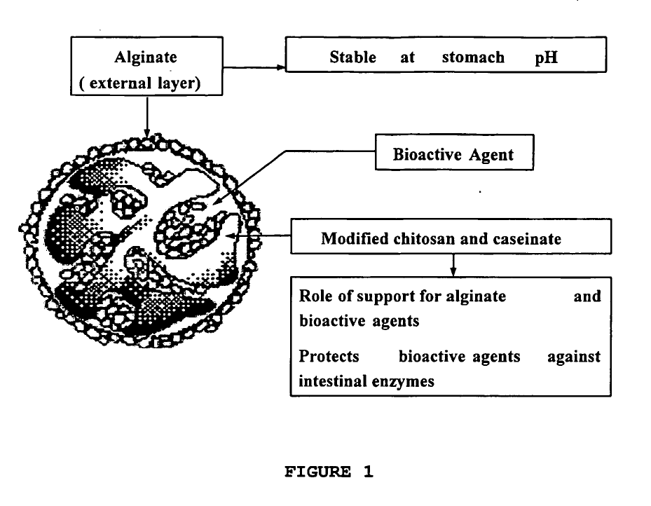

Based on the physical and chemical properties of the polymers, the structure of the spheres, according to our concept, may be a combination of several components (FIG. 1).

Modified alginate is thought to be at the exterior of the sphere to act as the envelope because of its resistance in an acid environment.

Modified and/or cross-linked chitosan is inside with the milk protein and the bioactive agent. The chitosan polymer precipitates and easily turns into a gel at neutral pH, incorporating the milk protein and bioactive agent into the matrix formed. The chitosan polymer's role is essentially to support the bioactive agent (e.g. an enzyme, a probiotic bacterium or a neutraceutic) and to protect it against degradation by attack from intestinal proteases.

The addition of milk protein reveals several advantages, particularly as an excellent nutritional source for the growth of lactic bacteria (in the case of probiotic formulation). Also, caseinate can retain calcium which is the main agent involved in ionotropic interactions of the alginate envelope (FIG. 1).

### Example 1

Derivatization of Chitosan and of Alginate by Acylation with Fatty Acids Residues and Cross-Linking with Bifunctional Agents.

Chitosan is derivatized with acid chlorides of fatty acids, giving amidic derivatives, involving part of the amino group of C2 of the aminoglucose units. Further cross-linking with dialdehydes occurs at the remaining (nonacylated) free amino groups.

IChitosan and alginate are modified with caproic acid or palmitic acid and cross-linked with glutaraldehyde. Derivitization of chitosan and alginate is done at pH 5.5-7.0 at a temperature of 60-100 degrees Celsius for 1-3 hours.

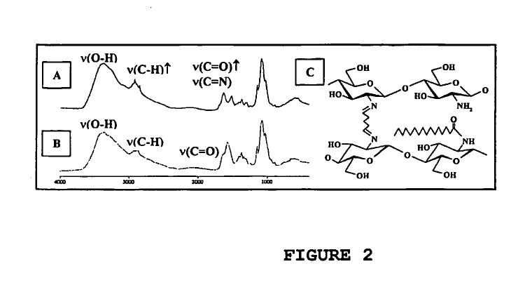

From Fourier transform infrared (FTIR) analysis (FIG. 2), it appears that acylation occurs first (coupling with fatty acids). An increase of the band in the 1700 cm⁻¹ spectral region appears after modification for the elongation vibration of the C═O groups. The same phenomenon is observed for the band at 2980 cm⁻¹, which might be due to the presence of C—H groups (presence of acyl chains from the fatty acids). Secondly, the formation of the imine bond (C═N) of the amine groups from chitosan and of carbonyl from dialdehyde is typical in the 1700 cm⁻¹ spectral region.

Alginate modification and cross-linking may be done in the same manner as chitosan. However, alginate may also be modified directly with fatty acid chlorides (leading to esters) and with amino groups introduced by previous derivatization with chloroethylamine at pH 9.5-11.0.

Various ratios of fatty acid chloride/chitosan and fatty acid chloride/alginate may be used to modify mechanical and release properties of the resulting products. Various fatty acid derivatives (from propionate (C3) up to stearate (C18)) may also be used to modify release and mechanical properties of the formulations.

### Example 2

Beads and Microparticles Based on Modified Chitosan, Alginate, and Milk Proteins, Including Pharmaceutical and Nutraceutical Agents

The presence of calcium caseinate creates a microenvironment where different degree of ionotropic gelation is observed inside the microparticles. Whey proteins can be added as a source of nutrient for probiotic bacteria.

Chitosan modified with caproic acid was dissolved (2-3%) in slightly acidic medium (pH 5.0-6.5) and mixed with lactic bacteria (or other active agent) solutions in presence of milk proteins (0.2-1% caseinate, rich in calcium). Beads are formed in solutions of tripolyphosphate, sedimented, recovered, suspended in native or modified alginate (1-3%), for various intervals and then, the medium was dripped in 5-10% CaCl₂, forming alginate beads.

### Example 3

Formulation of Therapeutic Enzymes within Chitosan/Alginate Microparticles

Catalase (EC 1.11.1.6) is an enzyme (240 kDa) that catalyzes the decomposition of hydrogen peroxide. Therapeutic forms of catalase are of interest for treating infections via intra-peritoneal administration.

To prepare a therapeutic formulation of this enzyme, catalase is formulated into polymeric spheres and the efficiency of such a beaded matrix is evaluated by determining catalytic activity (i.e. kinetic analysis of H₂O₂ decomposition by spectrophotometric measurement of ΔA/min at 240 nm).

Carbohydrate (alginate, chitosan and their derivatives) activated by treatment with Na-periodate chains for 3-12 hours generate carboxylic groups that bind enzymes via the e-amino group of the lysine residues in the enzyme. First, catalase is immobilized on alginate activated by Na-periodate activation. The alginate-catalase conjugate solution is dripped into 5-10% CaCl₂ solution for ionotropic gelation. A final treatment with chitosan blocks the excess carbonyl groups and, at the same time, reinforces the particles.

In this example, caproic acid is used to modify the chitosan and alginate. The results show that the apparent activity of immobilized catalase diminishes by about 50% in comparison with that of free enzyme. The loss is likely due to a transfer phenomenon, related to the diffusion of the substrate from the external environment to the enzyme and then of the enzyme product to the external environment. Although the enzyme is protected in the modified or cross-linked matrix from gastric and intestinal degradations, steric hindrance and diffusion phenomena can occur. Consequently, a higher matrix efficiency requires a polymer porosity large enough for the diffusion of the substrate and products through the semi-permeable matrix material. On the other hand, the results show that the activity of the immobilized catalase on the modified matrix is greater (40%) than in the free catalase (for which the loss of activity seems due to the catalase degradation in the gastric or intestinal phase, either by the acidity or by the proteases action).

Alternatively, enzymes can be immobilized in an alginate matrix that has been cross-linked via the action of glutaraldehyde (0.001-0.005%) on the amino groups introduced in alginate by previous derivatization with chloroethylamine.

### Example 4

Inclusion of Probiotics (Lactic Bacteria) into Modified Chitosan/Alginate Beads.

In order to evaluate matrix efficiency in the gastro-intestinal system, Lactobacillus plantarum, L. Rhamnosus, S. Thermophilus or other probiotics such as the commercial mixture called Bio K Plus” may be formulated using modified chitosan/alginate beads. In this example, Lactobacillus plantarum is used due to its sensitivity at pH<3.0.

Solutions of 2-3% alginate modified with caproic acid are mixed with a solution of whey proteins (0.2-2.0%) and with the medium containing lactic bacteria. The suspension is dripped into a solution of 5-10% CaCl₂, under stirring. The same preparation is done by using 2-3% modified chitosan mixed with whey proteins or Ca-caseinate and lactic bacteria. The suspension is dripped into a solution of 1-2% alginate, under stirring. The bead structure is shown in FIG. 1.

The preliminary results with L. Plantarum show a growth of the bacteria after 30 minutes in the gastric phase (pH=1.5 in the presence of pepsin) and after 24 hours in the intestinal phase (pH=7.0 in the presence of pancreatin).

Viability of microorganisms is confirmed on culture Man, Rogosa and Sharpe (MRS) medium, at 37 C.

### Example 5

Monolithic Dosage forms with Controlled Drug Release Based on Alginate, for Oral Administration

Modified alginate and derivatives are dried by acetone treatment, at gradually increasing concentrations.

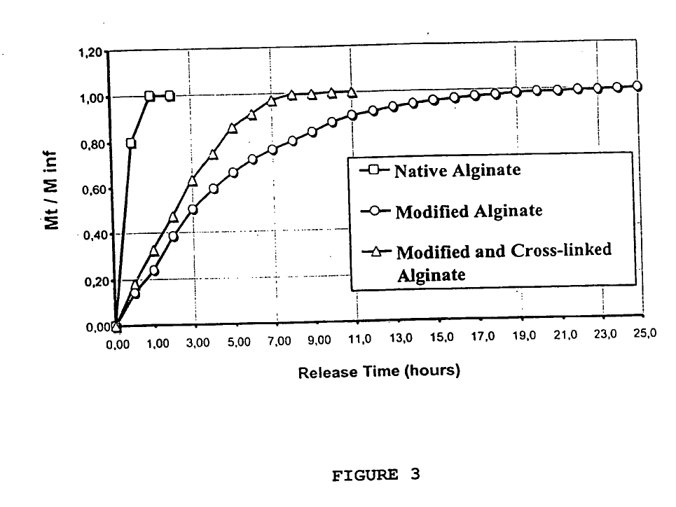

Tablets of 500 mg modified alginate (coupled with fatty acids and/or cross-linked by a dialdehyde) containing 20% of active tracer (i.e. 100 mg acetaminophen), are tested in an aqueous medium (pH 7, 37 C, 50 rpm) with a dissolution apparatus (Distek™) using a USP XXII method. For the alginate-based tablets, the derivatized and cross-linked polymer shows the best results. FIG. 3 shows the results when caproic acid is used to modify the alginate and glutaraldehyde is used to cross-link the alginate. The release of the therapeutic agent from this matrix is complete after 18 hours compared with the release of the same therapeutic agent from tablet based on native alginate (1 hour) or on alginate derivatized only (and not cross-linked) with 8 hours release time.

### Example 6

Monolithic Dosage forms with Controlled Drug Release Based on Chitosan Derivatives, as Implants for Subcutaneous Administration.

Modified chitosan derivatives are dried by acetone treatment, at gradually increasing concentrations.

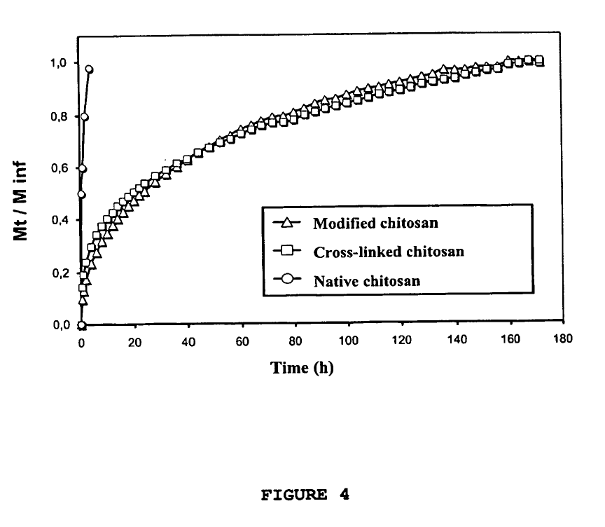

For study comparison, the same tablet size, weight and drug loading as in Example 5 were kept. Thus, tablets of 500 mg modified chitosan (coupled with fatty acids and/or cross-linked by a dialdehyde) containing 20% of active tracer (i.e. 100 mg acetaminophen), are tested in an aqueous medium (pH 7, 37 C, 50 rpm) with a dissolution apparatus (Distek™) using a USP XXII method. The results show, unexpectedly, a very slow controlled release of the active agent for a period of 160 hours. FIG. 4 shows the results when palmitic acid is used to modify the chitosan and glutaraldehyde is used the cross-link the chitosan. No significant differences are noticed between the two formulations—one with cross-linked chitosan only and one with chitosan that is both modified with fatty acid and cross-linked.

Although release times longer than 24 h are less useful for oral administration, the result is of great interest for the use of formulations based on modified chitosan as implants. Interest in implants is very high for human and veterinarian therapeutics, such as in the sub-cutaneous administration of antibiotics, steroids, peptide hormones, anticontraceptives, modulators of ovulation, etc.

Therefore, the formulations of this example are highly recommended for formulations of implants or transdermic patches.

### Example 7

Films Based on Modified Chitosan, Alginate, and Milk Proteins, including Pharmaceutical Agents

Modified chitosan (sol. 1-3%, pH 5.5-6.5) and alginate (sol. 1.5-3.0%, pH 6.5-7.5) may be used to generate films by casting. Both modified polymers were obtained as described in Example 1. The puncture strength (PS) of chitosan is approximately 550 N/mm, but no elasticity is noticed. The addition of fatty acids (functionalization agents) to the carbohydrate structure, improves not only the hydrophobicity but also the elasticity of the films. Due to their long hydrophobic chains, the fatty acids can be inserted between the chitosan macromolecular chains, thereby diminishing the intermolecular hydrogen interactions and bringing more flexibility, thus acting as plasticisers. The viscoelasticity coefficient is 0.68.

Although the PS is largely diminished during the acylation (from 550 to 150 N/mm), this biomembrane is resistant enough to be used as wrapper.

Similarly, for the alginate-based films the initial PS is 450 N/mm and after-acylation was 145 N/mm. No increase in elasticity is noticed. This may be explained by the presence of ionic interactions of the carboxyl groups from the alginate (at the C₆ level), except for the hydrogen bonds that are largely broken by the fatty acids. This also may explain why there is no significant difference in regard to the viscoelasticity coefficient, which is 0.44.

The invention being thus described, it is apparent to one skilled in the art that variations and modifications are possible and that such variations and modifications are intended to be included within the scope of the following claims.

1-30. (canceled)

31. A biocompatible carrier composition comprising a biocompatible carbohydrate polymer, wherein said biocompatible carbohydrate polymer is modified with a hydrophobic group.

32. The composition according to claim 31, wherein the carbohydrate polymer is a polysaccharide.

33. The composition according to claim 32, wherein the polysaccharide is selected from (a) chitosan, (b) alginate and (c) both (a) and (b).

34. The composition according to claim 31, wherein the hydrophobic group is a residue of a fatty acid.

35. The composition according to claim 34, wherein the fatty acid has from three to eighteen carbon atoms.

36. The composition according to claim 35, wherein the fatty acid is selected from palmitic acid, lauric acid, oleic acid, linoleic acid, linolenic acid, caproic acid, caprylic acid, stearic acid, propionic acid and butyric acid.

37. The composition according to claim 31, wherein the carbohydrate polymer is further cross-linked.

38. The composition according to claim 37, wherein the carbohydrate polymer is further cross-linked by a dialdehyde, epichlorohydrin, ethylchloroformate or phosphorus oxychloride.

39. The composition according to claim 38, wherein the dialdehyde is glutaraldehyde.

40. The composition according to claim 31 further comprising a milk protein.

41. The composition according to claim 40, wherein the milk protein is selected from (a) a caseinate, (b) a whey protein and (c) both (a) and (b).

42. The composition according to claim 31 further comprising a bioactive agent.

43. The composition according to claim 42, wherein the bioactive agent is selected from a drug, a vitamin, a mineral, bacteria, a bacteriocine, an anti-oxidant and an anti-microbial.

44. The composition according to claim 43 which is in the form selected from the group consisting of a tablet, an implant, a microsphere and a film.

45. A method of controlling the release of a bioactive agent into an environment comprising:
(a) formulating the bioactive agent in the composition according to claim 31; and
(b) administering to the environment the composition containing the bioactive agent.

46. A method of formulating a bioactive agent comprising the step of coating, encapsulating or incorporating the bioactive agent with the composition according to claim 31.

47. A method of preparing a pharmaceutical formulation comprising coating, encapsulating or incorporating a bioactive agent with the composition according to claim 31.

48. The method according to claim 47, wherein the pharmaceutical formulation is in the form selected from a tablet, an implant, a microsphere and a film.

49. A method for packaging food comprising the step of coating food with the composition according to claim 31.

50. The method according to claim 49, wherein the coating is in the form of a film.
